(19) United States
(12) Patent Application Publication
(10) Pub. No.: US 2026/0124353 A1
(43) Pub. Date: May 7, 2026

(54) SAFETY CONTROL FOR NEGATIVE PRESSURE WOUND THERAPY DEVICES

(71) Applicant: T.J.Smith and Nephew,Limited, Hull (GB)

(72) Inventors: David Michael Elder, Hull (GB); Felix Clarence Quintanar, Hull (GB)

(21) Appl. No.: 19/116,306

(22) PCT Filed: Dec. 18, 2023
(86) PCT No.: PCT/EP2023/086440

§ 371 Date: Mar. 27, 2025

(30) Foreign Application Priority Data
Dec. 23, 2022 (GB) 2219619.0

(51) Int. Cl. A61M 1/00

(52) U.S. Cl. CPC A61M 1/964; A61M 2205/502; A61M 2205/702; A61M 2205/8206

(57) ABSTRACT

A negative pressure wound therapy device can include a negative pressure source configured to provide, via a fluid flow path, negative pressure therapy to a wound covered by a wound dressing. The device can include electronic circuitry that includes a first processor configured to operate the negative pressure source and a second processor configured to operate a user interface and control the first processor. The second processor can be configured to, in response to detecting an error in operation of the first processor or the negative pressure source, reset the first processor or the negative pressure source and increment a first counter. Resetting the first processor or the negative pressure source can cause provision of the negative pressure therapy to be restored. In response to a determination that the first counter satisfies a first threshold, the second processor can deactivate provision of the negative pressure therapy.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy device can include a negative pressure source configured to provide, via a fluid flow path, a negative pressure therapy to a wound covered by a wound dressing. The device can include an electronic circuitry. The electronic circuitry can include a user interface configured to permit a user to adjust one or more parameters of the negative pressure therapy. The electronic circuitry can include a first processor configured to operate the negative pressure source. The electronic circuity can include a second processor that can be configured to operate the user interface and control the first processor. The second processor can be configured to, in response to detecting an error in an operation of the first processor or the negative pressure source, reset the first processor or the negative pressure source and increment a first counter. Resetting the first processor or the negative pressure source can cause provision of the negative pressure therapy to be restored. The second processor can be configured to, in response to a determination the first counter satisfies a first threshold, deactivate provision of the negative pressure therapy.

The negative pressure wound therapy device of any of the preceding paragraphs and/or any of the devices, apparatuses, or systems disclosed herein can include one or more of the following features. Resetting the first processor or the negative pressure source can include disconnect the first processor or the negative pressure source from a power source.

The negative pressure wound therapy devices of any of the preceding paragraphs and/or any of the devices, apparatuses, or systems disclosed herein can include one or more of the following features. The second processor can be configured to, in response to detecting the error in the operation of the first processor or the negative pressure source, provide an indication on the user interface. The second processor can be configured to detect the error in the operation of the first processor or the negative pressure source in response to a detection of an error in a negative pressure source activity signal. The negative pressure source activity signal can be a rotation speed of a motor of the negative pressure source.

The negative pressure wound therapy device of any of the preceding paragraphs and/or any of the devices, apparatuses, or systems disclosed herein can include one or more of the following features. The second processor can be configured to reset the first counter in response to not detecting the error in the operation of the first processor or the negative pressure source over a duration of time. The second processor can be configured to, in response to detecting an error in an operation of the user interface, reset the user interface and increment a second counter. The second processor can be configured to, in response to a determination the second counter satisfies a second threshold, deactivate provision of the negative pressure therapy. Resetting the user interface can include disconnecting the user interface from a power source. The user interface can include a display with a backlight. The second processor can be configured to detect the error in the operation of the display in response to monitoring voltage or current of the backlight.

The negative pressure wound therapy device of any of the preceding paragraphs and/or any of the devices, apparatuses, or systems disclosed herein can include one or more of the following features. The device can include a power source and a power source circuitry configured to monitor the power source. The second processor can be configured to, in response to detecting an error in an operation of the power source circuitry, reset the power source circuitry or the power source and increment a second counter. The second processor can be configured to, in response to a determination the second counter satisfies a second threshold, deactivate provision of the negative pressure therapy. Resetting the power source circuitry can include disconnecting the power source circuitry from a power source.

A non-transitory computer readable medium can store instructions that, when executed by a first processor of a negative pressure wound therapy device, cause the first processor to, in response to detecting an error in an operation of 1) a second processor configured to operate a negative pressure source or 2) the negative pressure source, reset the second processor or the negative pressure source and incrementing a first counter. Resetting the second processor or the negative pressure source can cause provision of a negative pressure therapy to be restored. The negative pressure therapy can be provided by the negative pressure source to a wound covered by a wound dressing. The instructions can cause the first processor to, in response to determining that the first counter satisfies a first threshold, deactivate provision of the negative pressure therapy.

The computer readable medium of any of the preceding paragraphs and/or any of the media disclosed herein can include one or more of the following features. Resetting the second processor or the negative pressure source can include disconnecting the second processor or the negative pressure source from power. The instructions can cause the first processor to, in response to detecting the error in the operation of the second processor or the negative pressure source, provide an indication on a user interface configured to permit a user to adjust one or more parameters of the negative pressure therapy. The instructions can cause the first processor to detect the error in the operation of the second processor or the negative pressure source in response to detecting an error in a negative pressure source activity signal. The negative pressure source activity signal can be a rotation speed of a motor of the negative pressure source.

The computer readable medium of any of the preceding paragraphs and/or any of the media disclosed herein can include one or more of the following features. The instructions can cause the first processor to reset the first counter in response to not detecting the error in the operation of the first second or the negative pressure source over a duration of time. The instructions can cause the processor to, in response to detecting an error in an operation of a user interface configured to permit a user to adjust one or more parameters of the negative pressure therapy, reset the user interface and increment a second counter. The instructions can cause the first processor to, in response to determining that the second counter satisfies a second threshold, deactivate provision of the negative pressure therapy. The user interface can include a display with a backlight. The instructions can cause the first processor to detect the error in the operation of the display in response to monitoring voltage or current of the backlight. Resetting the user interface can include disconnecting the user interface from a power source.

The computer readable medium of any of the preceding paragraphs and/or any of the media disclosed herein can include one or more of the following features. The instructions can cause the processor to, in response to detecting an error in an operation of a power source circuitry configured to operate a power source, reset the power source circuitry or the power source and increment a second counter. The instructions can cause the processor to, in response to determining that the second counter satisfies a second threshold, deactivate provision of the negative pressure therapy. Resetting the power source circuitry can include disconnecting the power source circuitry from the power source.

A negative pressure wound therapy device can include a negative pressure source configured to provide, via a fluid flow path, negative pressure to a wound covered by a wound dressing. The device can include a user interface configured to receive input from a user and provide at least one indication to the user. The device can include a main control circuitry configured to operate the negative pressure source and the user interface. The device can include a power source configured to provide power to the negative pressure source, user interface, and the main control circuitry. The power source can include at least one rechargeable battery and a power source control circuitry powered by the at least one rechargeable battery. the power source control circuitry can be configured to operate the power source in a sleep mode in which a first level of power is provided from the at least one rechargeable battery to one or more of the negative pressure source, user interface, or the main control circuitry. The power source control circuitry can be configured to operate the power source in a normal mode in which a second level of power is provided from the at least one rechargeable battery to one or more of the negative pressure source, user interface, or the main control circuitry. The second level of power can be greater than the first level of power. The second level of power can be sufficient to cause the negative pressure source to be activated. The power source control circuitry can be configured to, while operating the power source in the sleep mode, verify that at least one of a user input has been received from the user interface or an external power supply has been connected. The power source control circuitry can be configured to, in response to verifying that at least one of the user input has been received from the user interface or the external power supply has been connected, operate the power source in the normal mode.

The negative pressure wound therapy device of any of the preceding paragraphs and/or any of the devices, apparatuses, or systems disclosed herein can include one or more of the following features. The power source control circuitry can be configured to, in response to not verifying that at least one of the user input has been received from the user interface or the external power supply has been connected, continue operating the power source in the sleep mode.

The negative pressure wound therapy device of any of the preceding paragraphs and/or any of the devices, apparatuses, or systems disclosed herein can include one or more of the following features. The first level of power can be insufficient to cause the negative pressure source to be activated. The user input can include a press of a button associated with start of provision of negative pressure.

The negative pressure wound therapy device of any of the preceding paragraphs and/or any of the devices, apparatuses, or systems disclosed herein can include one or more of the following features. The power source control circuitry can be configured to, while operating the power source in the sleep mode, verify over a duration of time that at least one of a user input has been received from the user interface or an external power supply has been connected. The power source control circuitry can be configured to, in response to verifying over the duration of time that at least one of the user input has been received from the user interface or the external power supply has been connected, operate the power source in the normal mode.

The negative pressure wound therapy device of any of the preceding paragraphs and/or any of the devices, apparatuses, or systems disclosed herein can include one or more of the following features. The power source control circuitry can be configured to, in response to not verifying over the duration of time that at least one of the user input has been received from the user interface or the external power supply has been connected, continue operating the power source in the sleep mode.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIG. 5 illustrates a process for safety control.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

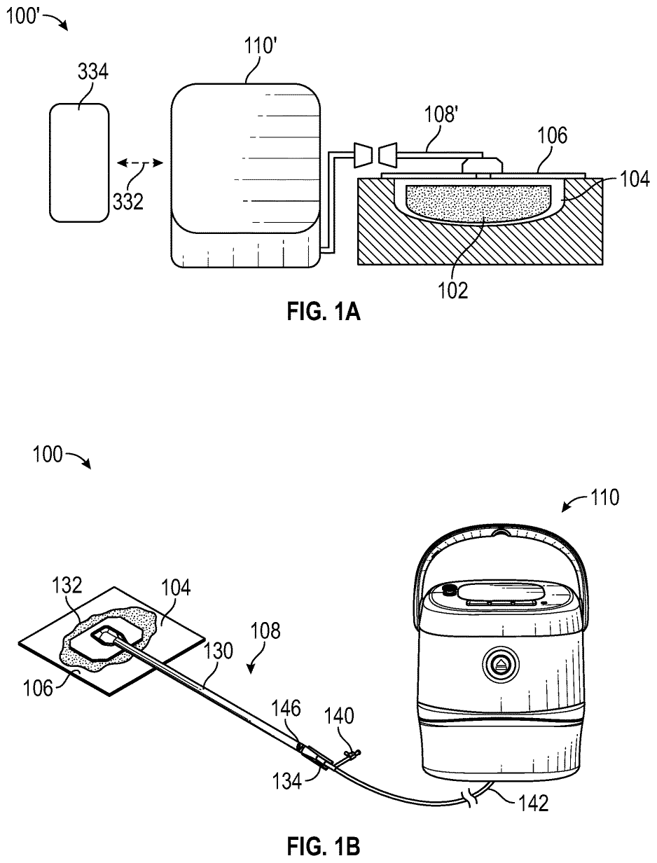

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

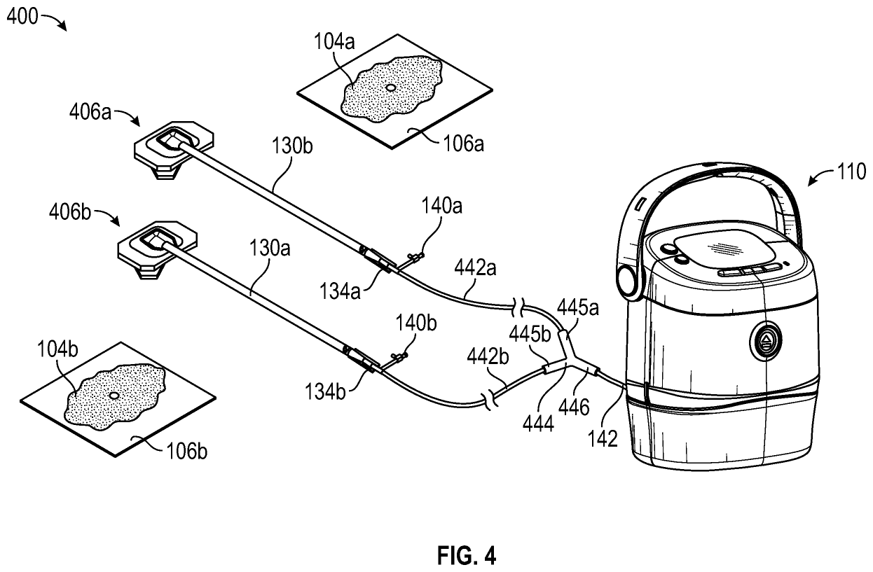

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970.RTM., or Gehring 879.RTM.) or a nonwoven material, or terry-woven or loop-pile materials. The fibers may not necessarily be woven, and can include felted and flocked (including materials such as Flotex.RTM.) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 um. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

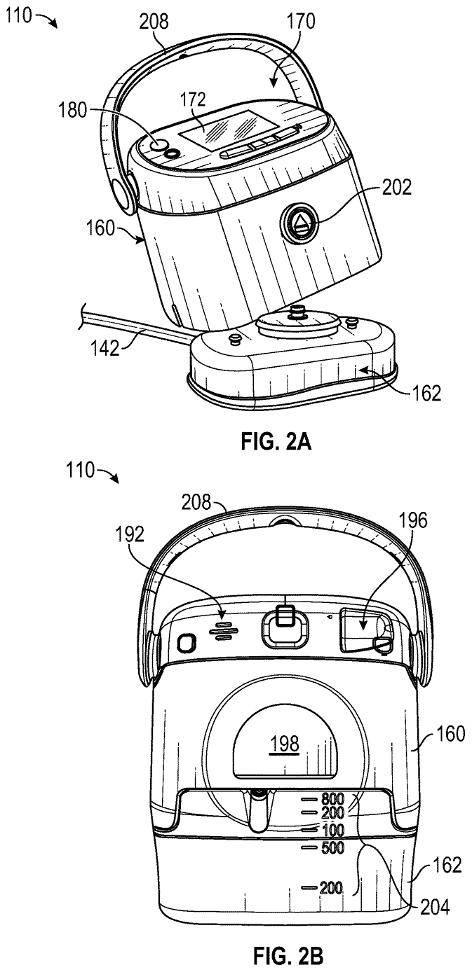

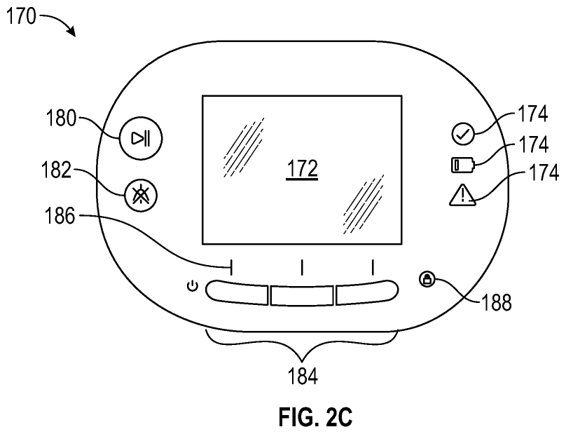

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

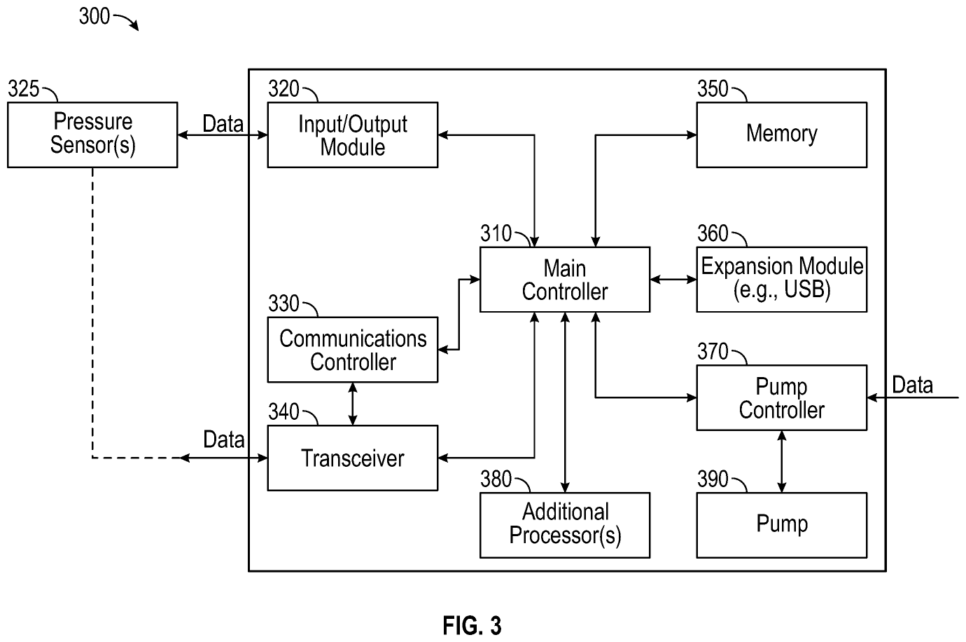

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110 can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850 or International Publication No. WO 2018/167199, each of which is incorporated by reference in its entirety.

### Integrated Negative Pressure Wound Therapy

In some implementations, a wound therapy device can be mounted on or otherwise supported by a wound dressing. A source of negative pressure (such as, a pump) and some or all other components of the wound therapy device, such as power source(s), sensor(s), connector(s), user interface component(s) (such as button(s), switch(es), speaker(s), screen(s), etc.) and the like, can be integral with the wound dressing. The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing. The wound dressing can be provided as a single article with all wound dressing elements (including the pump) pre-attached and integrated into a single unit.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site. Integrated wound therapy devices can additionally or alternatively include one or more features described in International Application No. PCT/EP 2018/074694, filed Sep. 13, 2018, titled “NEGATIVE PRESSURE WOUND TREATMENT APPARATUSES AND METHODS WITH INTEGRATED ELECTRONICS,” International Application No. PCT/EP2018/074701, filed Sep. 13, 2018, titled “NEGATIVE PRESSURE WOUND TREATMENT APPARATUSES AND METHODS WITH INTEGRATED ELECTRONICS,” International Application No. PCT/EP 2018/079345, filed Oct. 25, 2018, titled “NEGATIVE PRESSURE WOUND TREATMENT APPARATUSES AND METHODS W″ITH INTEGRATED ELECTRONICS,” and International Application No. PCT/EP2020/056317, filed Mar. 10, 2020, titled “EXHAUST BLOCKAGE DETECTION FOR NEGATIVE PRESSURE WOUND TREATMENT APPARATUSES,” each of which is incorporated by reference herein in its entirety

### Safety Control

One or more electronic components of any of the wound therapy devices disclosed herein (such as the device 110) may malfunction. Malfunction can be caused by a noisy environment, for instance, by exposure to electromagnetic interference (due to, for example, flying in an aircraft). While shutting down a wound therapy device responsive to detection of malfunction may be desirable from the perspective of patient safety, this approach would undesirably cause interruption in the provision of negative pressure wound therapy. Instead, it may be desirable to attempt to reset the electronic component that has been determined to malfunction in order to maintain the provision therapy. This approach can facilitate patient safety as well as serve the goal of providing therapy to the patient.

As described herein, a first processor (such as, a main controller 310) can be responsible for controlling the user interface, and a second processor (such as, a pump controller 370) can be responsible for controlling the pump 390. The first processor can monitor malfunction of the pump 390 or the pump controller 370. An example of malfunction detection would be detection of an incorrect activity signal (or absence of such signal at a time the signal would be expected) indicative of the activity of the pump 390. The activity signal can reflect the rotation speed of a motor of the pump 390 (which can be measured by a tachometer), duty cycle of the pump 390, or the like. The pump controller 370 can control the pump 390 based on the activity signal. The pump controller 370 can detect that the activity signal is incorrect (such as, negative, too small, or too large) or absent. In response to the detection of malfunction, the pump 390 can be reset. Resetting the pump 390 can be initiated by one or more of the main controller 310 or the pump controller 370. Resetting can include activating or toggling a reset pin (or reset line). If this is not successful (for instance, because stored charge needs to be discharged), resetting can include power cycling where power is disconnected for a period of time. In some cases, power cycling can be performed right away in response to detection of malfunction (such as, with an electronic component that does not have a reset pin or reset line).

Continuing with the example of detection of an incorrect activity signal or absence of such signal, the pump 390 can be reset (or power cycled). This can be performed by the first processor or the pump controller 370. In some implementations, the pump controller 370 can be additionally or alternatively reset (or power cycled), which can be performed by the main controller 310. As a result of resetting one of more of the pump 390 or the pump controller 370, negative pressure wound therapy can be continued (for instance, negative pressure set point can be reestablished at the wound). Advantageously, essential performance (which can be an operational mode with reduced therapy features) can be maintained even when malfunction has occurred.

Detection of a malfunction of the activity signal (or any other signal described herein) can include noise detection. Noise can be detected by performing a sweep across one or more frequency bands of interest and determining presence of energy in the one or more frequency bands. Presence of sufficient energy (such as, energy that satisfies an energy threshold) in one or more frequency bands in which energy due to a particular signal is not expected to be present can be due to noise and indicate malfunction. For example, presence of sufficient energy in one or more frequency bands in the Megahertz range can be indicative of malfunction of the activity signal.

To facilitate patient safety, the number of resets can be tracked, for instance, by incrementing a counter. The counter can be maintained in a non-volatile memory so that the counter value persists in case of a reset (or power cycle). If the number of resets satisfies (such as, reaches) a threshold (such as, 2, 3, 4, 5 or more), this can indicate that the malfunction likely cannot be corrected and may be fatal to the safe operation of the wound therapy device. Accordingly, therapy can be deactivated. In some cases, therapy can be permanently deactivated so that the pump 390 is prevented from being activated again (at least until the wound therapy device has been serviced by the manufacturer or a service professional).

To facilitate continuous provision of therapy, the counter can be reset. The counter can be reset responsive to not detecting a malfunction over a period of time. For example, responsive to a normal operation of one of more of the pump 390 or the pump controller 370 over a threshold duration of time (such as, 1 minute, 2 minutes, 3 minutes, 4 minutes, 5 minutes or more), the counter can be reset.

Determination of an unsafe level of negative pressure at the wound (or overpressure) can lead to immediate deactivation of the pump 390. This can be performed in order to maintain patient safety as overpressure can cause pain or otherwise be detrimental to the patient's health.

In some instances, the power source can be a smart battery pack (or a smart power supply) with an electronic circuitry that monitors and controls charging and discharging of the power source (which can include one or more batteries that can be rechargeable). The electronic circuitry can a power source controller that can vary the charging current according to one or more of several operational parameters of the one or more batteries. These operational parameters can include the charge level, temperature, number of charge cycles of the one or more batteries, age of the one or more batteries, or another metrics used to quantify battery performance. Monitoring the one or more operational parameters can facilitate determining the accurate charge level, determining and providing an indication of battery fuel gauge (which is associated with the remaining estimated charge), providing charging control, protecting the one or more batteries, or the like. Smart battery pack one can additionally or alternatively include one or more features described in International Patent Application No. PCT/EP 2022/083496, filed Nov. 28, 2022 and published as WO 2023/110376, titled “Smart Battery Pack with Power Saving Modes for Negative Pressure Wound Therapy Devices,” which is incorporated by reference in its entirety.

Operation of a smart battery pack can be similarly monitored and, if a malfunction is detected, the smart battery pack can be reset (or power cycled). In some cases, the smart battery pack can communicate with the main controller 310 over I2C bus.

In some cases, power can be supplied by an external power supply and the device may not have sufficient control to reset (or power cycle) the external power supply. Still, the supplied power signal can be monitored. If it is determined that the supplied power signal is noisy, which can indicate malfunction of the external power supply, an indication can be provided to the user to check the connection to the external power supply or check the external power supply. The indication can be one or more of a visual, audible, or tactile indication.

Operation of the user interface can similarly be monitored and, if a malfunction is detected, the component can be reset (or power cycled). For example, the display 172 can be an LCD screen with a backlight. Current or voltage on one or more electrical connections for the backlight can be monitored to detect a malfunction of the display 172. This can be performed by monitoring the voltage or current across in electrical communication with the one or more electrical connections for the backlight.

Operation of other electronic components can be similarly monitored and, if a malfunction is detected, the component can be reset (or power cycled). For example, malfunction or one or more pressure sensors 325 can be detected. Malfunction can be detected based on an incorrect pressure reading (such as, too small or too large) or absence of a pressure reading over a period of time. As another example, operation of one or more for I/O module 320, communications controller 330, transceiver 340, memory 350, expansion module 360, or one or more additional processors 380 can be similarly monitored and, if malfunction is detected, the component can be reset (or power cycled). Malfunction can be determined responsive to receiving incorrect data or not receiving data from the particular electronic component.

Communication with any electronic component being monitored can be performed using serial (for example, I2C), parallel, hybrid communication protocol. A plurality of counters can be maintained for the plurality of electronic components being monitored. A plurality of thresholds for the plurality of counters can be utilized. As described herein, any of the counters can be reset responsive to not detecting a malfunction of a particular electronic component over a period of time.

Resetting (or power cycling) caused by the detection of malfunction of an electronic component can be indicated to the user. For example, one or more of a visual, audible, or tactile indication can be provided using any of the approaches described herein. Providing an indication can help the user understand, for example, why therapy has been interrupted.

Malfunction of the main controller 310 can be monitored by another controller (such as, the pump controller 370) or a dedicated circuitry (for instance, watchdog circuitry). If a malfunction is detected, the main controller 310 can be reset (or power cycled) as described herein.

Provision of negative pressure wound therapy may not need to be interrupted responsive to the detection of malfunction of an electronic component that does not participate in the delivery of therapy. Such electronic components can include one or more of the display 172, communications controller 330, transceiver 340, memory 350, expansion module 360, main controller 310, or the like.

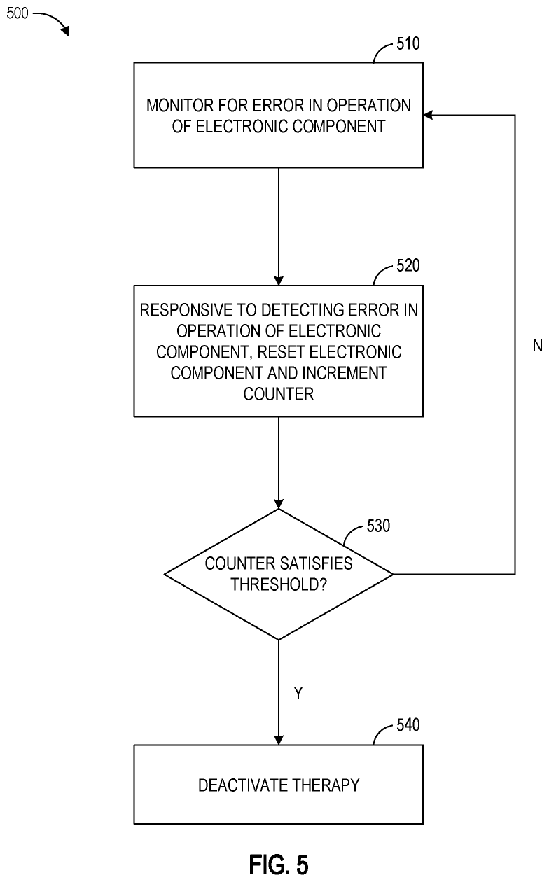

FIG. 5 illustrates a safety control process 500. The process 500 can be implemented by any wound therapy device disclosed herein. For example, the process 500 can be implemented by a controller of the wound therapy device, such as the main controller 310. In block 510, the process 500 can monitor operation of one or more electronic components of the wound therapy device and determine whether there are any errors in the operation. If an error is detected, the process 500 can transition to block 520 where the process can reset the electronic component. As described herein, resetting can include power cycling the electronic component. In block 520, the process can increment a counter. In block 530, the process 500 can compare the counter to a threshold. If the counter does not satisfy the threshold, the process 500 can transition back to block 510. If the counter satisfies the threshold, the process 500 can deactivate therapy in block 540.

Advantageously, the disclosed approaches for safety control allow for individually resetting (or power cycling) electronic components to maintain essential performance without comprising patient safety, even when operating in a noisy environment.

### Power Management

As described herein, the power source can be a smart battery pack that can alternate between several operating modes. The power source can include electronic circuitry, such as a power source controller, that can implement transition between the operating modes. In a normal operating mode, the power source controller can facilitate normal provision of power (such as, current) from one or more batteries of the power source to other electric components of a wound therapy device (for instance, the wound therapy device 110), such as to the negative pressure source. Normal operating mode can be initiated when the wound therapy device is being used for providing therapy to a patient. In a sleep operating mode, the power source controller can reduce the rate of power consumption from the one or more batteries. Sleep operating mode can be useful when the wound therapy device is in storage at a medical facility and awaiting to be used on a patient. Capacity of the power source can be conserved in the sleep mode so that the wound therapy device would be ready to provide therapy to the patient when called upon without needing to be charged. Some amount of power (lesser than in the normal operating mode) can be provided by the power source in the sleep operating mode, such as to permit the main controller 310 to operate. In some instances, a shutdown operating mode can be implemented in which provision of power by the one or more batteries can be disabled by the power source controller. The shutdown operating mode can allow the wound therapy device to remain inactive for long periods of time (such as, in a wound therapy device's manufacturing facility or in transit) while preserving the charge level of the one or more batteries. In the shutdown operating mode, the wound therapy device may not respond to user input because the main controller 310 does not receive any power from the power source.

The power source controller can transition to the power source between the operating modes. For instance, a transition from the shutdown operating mode to the sleep (or normal) operating mode can be made responsive to an external power supply (or charger) being connected to the wound therapy device, such as being connected to the power jack 196. As another example, a transition from the sleep operating mode to the normal operating mode can be made responsive to receiving a user input, such as responsive to a press of the button 202.

As described herein, smart battery pack can additionally or alternatively include one or more features described in International Publication No. WO 2023/110376, which is incorporated by reference in its entirety.

In some cases, presence of noise (such as, electromagnetic inference) may be mistakenly interpreted by the wound therapy device as a user input indicating a transition from the sleep operating mode to the normal operating mode. It can be advantageous for the wound therapy device to distinguish “true” events that trigger transition to the normal mode from spurious events caused by noise. The wound therapy device (such as, via one or more of the power source controller or the main controller 310) can verify that the transition from the sleep operating mode to the normal operating mode was intended by verifying that the user input was received (such as, the button 202 was pressed). If so, transition to the normal operating mode would be made. Otherwise, the power source can be caused to remain in the sleep operating mode. In certain implementations, a transition from the normal operating mode to the sleep operating mode can be caused by connecting the external power supply to the wound therapy device. In such cases, the wound therapy device can similarly verify that the transition from the sleep (or shutdown) operating mode to the normal operating mode was intended by verifying that the external power supply has been connected.

Filtering can be additionally performed to ensure that the normal operating mode is not triggered by spurious noise burst that may be interpreted as the user input or connection of the external power supply. This can be achieved by verifying one or more of receipt of user input or connection of the external power supply multiple times (such as, over a duration of time).

Advantageously, the disclosed approaches for power management allow for preserving power source capacity when the wound therapy device is not in use, even when operating in a noisy environment.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy device comprising:
a negative pressure source configured to provide, via a fluid flow path, a negative pressure therapy to a wound covered by a wound dressing; and
an electronic circuitry comprising:
a user interface configured to permit a user to adjust one or more parameters of the negative pressure therapy;
a first processor configured to operate the negative pressure source; and
a second processor configured to operate the user interface and control the first processor, the second processor further configured to:
in response to detecting an error in an operation of the first processor or the negative pressure source, reset the first processor or the negative pressure source and increment a first counter, wherein resetting the first processor or the negative pressure source causes provision of the negative pressure therapy to be restored; and
in response to a determination that the first counter satisfies a first threshold, deactivate provision of the negative pressure therapy.

2. The negative pressure wound therapy device of claim 1, wherein resetting the first processor or the negative pressure source comprises disconnecting the first processor or the negative pressure source from a power source.

3. The negative pressure wound therapy device of claim 1, wherein the second processor is further configured to, in response to detecting the error in the operation of the first processor or the negative pressure source, provide an indication of the error on the user interface.

4. The negative pressure wound therapy device of claim 1, wherein the second processor is configured to detect the error in the operation of the first processor or the negative pressure source in response to detecting an error in a negative pressure source activity signal.

5. The negative pressure wound therapy device of claim 4, wherein the negative pressure source activity signal comprises a rotational speed of a motor of the negative pressure source.

6. The negative pressure wound therapy device of claim 1, wherein the second processor is configured to reset the first counter in response to not detecting the error in the operation of the first processor or the negative pressure source over a duration of time.

7. The negative pressure wound therapy device of claim 1, wherein the second processor is further configured to:
in response to detecting an error in an operation of the user interface, reset the user interface and increment a second counter; and
in response to a determination the second counter satisfies a second threshold, deactivate provision of the negative pressure therapy.

8. The negative pressure wound therapy device of claim 7, wherein the user interface comprises a display with a backlight, and wherein the second processor is configured to detect the error in the operation of the display in response to monitoring voltage or current of the backlight.

9. The negative pressure wound therapy device of claim 1, wherein resetting the user interface comprises disconnecting the user interface from a power source.

10. The negative pressure wound therapy device of claim 1, further comprising a power source and a power source circuitry configured to monitor the power source, wherein the second processor is further configured to:
in response to detecting an error in an operation of the power source circuitry, reset the power source circuitry or the power source and increment a second counter; and
in response to a determination the second counter satisfies a second threshold, deactivate provision of the negative pressure therapy.

11. The negative pressure wound therapy device of claim 10, wherein resetting the power source circuitry comprises disconnecting the power source circuitry from the power source.

12. (canceled)

13. A non-transitory computer readable medium storing instructions that, when executed by a first processor of a negative pressure wound therapy device, cause the first processor to:
in response to detecting an error in an operation of 1) a second processor configured to operate a negative pressure source or 2) the negative pressure source, reset the second processor or the negative pressure source and incrementing a first counter, wherein resetting the second processor or the negative pressure source causes provision of a negative pressure therapy to be restored, the negative pressure therapy being provided by the negative pressure source to a wound covered by a wound dressing; and
in response to determining that the first counter satisfies a first threshold, deactivate provision of the negative pressure therapy.

14. The computer readable medium of claim 13, wherein resetting the second processor or the negative pressure source comprises disconnecting the second processor or the negative pressure source from power.

15. The computer readable medium of claim 13, wherein the instructions further cause the first processor to:
in response to detecting the error in the operation of the second processor or the negative pressure source, provide an indication on a user interface configured to permit a user to adjust one or more parameters of the negative pressure therapy.

16. The computer readable medium of claim 13, wherein the instructions further cause the first processor to detect the error in the operation of the second processor or the negative pressure source in response to detecting an error in a negative pressure source activity signal.

17. (canceled)

18. The computer readable medium of claim 13, wherein the instructions cause the first processor to reset the first counter in response to not detecting the error in the operation of the second processor or the negative pressure source over a duration of time.

19. The computer readable medium of claim 13, wherein the instructions further cause the first processor to:
in response to detecting an error in an operation of a user interface configured to permit a user to adjust one or more parameters of the negative pressure therapy, reset the user interface and increment a second counter; and
in response to determining that the second counter satisfies a second threshold, deactivate provision of the negative pressure therapy.

20. The computer readable medium of claim 19, wherein the user interface comprises a display with a backlight, and wherein the instructions cause the first processor to detect the error in the operation of the display in response to monitoring voltage or current of the backlight.

21. (canceled)

22. The computer readable medium of claim 13, wherein the instructions further cause the first processor to:
in response to detecting an error in an operation of a power source circuitry configured to operate a power source, reset the power source circuitry or the power source and increment a second counter; and
in response to determining that the second counter satisfies a second threshold, deactivate provision of the negative pressure therapy.

23. (canceled)

24. A negative pressure wound therapy device comprising:
a negative pressure source configured to provide, via a fluid flow path, negative pressure to a wound covered by a wound dressing;
a user interface configured to receive input from a user and provide at least one indication to the user;
a main control circuitry configured to operate the negative pressure source and the user interface; and
a power source configured to provide power to the negative pressure source, user interface, and the main control circuitry, the power source comprising at least one rechargeable battery and a power source control circuitry powered by the at least one rechargeable battery, the power source control circuitry configured to:
operate the power source in a sleep mode in which a first level of power is provided from the at least one rechargeable battery to one or more of the negative pressure source, user interface, or the main control circuitry;
operate the power source in a normal mode in which a second level of power is provided from the at least one rechargeable battery to one or more of the negative pressure source, user interface, or the main control circuitry, the second level of power being greater than the first level of power, and the second level of power being sufficient to cause the negative pressure source to be activated;
while operating the power source in the sleep mode, verify that at least one of a user input has been received from the user interface or an external power supply has been connected; and
in response to verifying that at least one of the user input has been received from the user interface or the external power supply has been connected, operate the power source in the normal mode.

25. (canceled)
